(19) United States
(12) Patent Application Publication
(10) Pub. No.: US 2020/0216496 A1
(43) Pub. Date: Jul. 9, 2020

(54) Antibacterial Compounds Produced by the Microorganism Strain Corallococcus Coralloides ST201330 (DSM 24989)

(71) Applicant: Sanofi, Paris (FR)

(72) Inventors: Heike Stump, Frankfurt (DE); Luigi Toti, Frankfurt (DE); Michael Kurz, Frankfurt (DE); Sabine Haag Richter, Oberursel (DE); Diertmar Schummer, Langen (DE)

(21) Appl. No.: 16/648,561

(22) PCT Filed: Sep. 17, 2018
(86) PCT No.: PCT/EP2018/075083

§ 371 Date: Mar. 18, 2020

(30) Foreign Application Priority Data
Sep. 18, 2017 (EP) 17306204.3

(51) Int. Cl. C07K 7/06; C12R 1/01

(52) U.S. Cl. CPC C07K 7/06; C12R 1/01

(57) ABSTRACT

The present invention relates to novel compounds of the formula (I), in which R is is hydrogen or hydroxy, that can be obtained by fermentation of the microorganism strain Corallococcus coralloides ST201330 (DSM 24989).

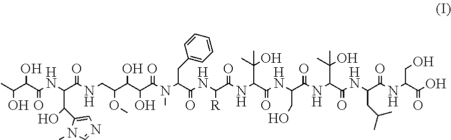

The present invention relates to novel compounds with antibacterial activities.

A large number of antibiotics are employed therapeutically for the treatment of bacterial infections. The causative pathogenic bacteria, however, are becoming increasingly resistant to the antibacterials used, and a great danger impends due to multi-resistant microorganisms that carry resistances not only against one antibacterial, but simultaneously against several groups of antibacterials. There are even Gram-negative bacteria that have become resistant to all commercially available antibacterials; as a consequence, infections that are caused by bacteria of this type are no longer treatable. Thus, there is a great need for novel agents that have antibiotic activity and suitable other properties such as low toxicity, for example, and that can be employed in the treatment of bacterial infections (cf., for example, H. W. Boucher et al., Clinical Infectious Diseases 2009, vol. 48, p. 1-12).

It has now been found that the microorganism strain Corallococcus coralloides ST201330 (DSM 24989) produces compounds, such as the compounds of the formula I defined thereafter, which have antibacterial activities against Gram-negative bacteria such as Salmonella typhimurium, Klebsiella pneumoniae, and Escherichia coli.

A subject of the present invention thus is a compound obtainable by fermentation of the microorganism strain ST201330 (DSM 24989) and isolation from the culture medium, for example under the conditions described further below herein or a pharmaceutically acceptable salt of any of them that has antibacterial activity, in particular against Gram-negative bacteria such as Escherichia coli. Another subject of the present invention is a compound of the molecular formula C₅₁H₈₁N₁₁O₂₁ or the molecular formula C₅₁H₈₁N₁₁O₂₂, which is obtainable by fermentation of the microorganism strain ST201330 (DSM 24989) and isolation from the culture medium, for example under the conditions described further below herein, or a pharmaceutically acceptable salt of any of them. Another subject of the present invention is a compound of the molecular formula C₅₁H₈₁N₁₁O₂₁ or of the molecular formula C₅₁H₈₁N₁₁O₂₂, which is obtainable by fermentation of the microorganism strain ST201330 (DSM 24989) and isolation from the culture medium, for example under the conditions described further below herein, or a pharmaceutically acceptable salt of any of them, which has antibacterial activity, in particular against gram-negative bacteria such as Escherichia coli, for example.

Another subject of the present invention is a compound of the formula I,

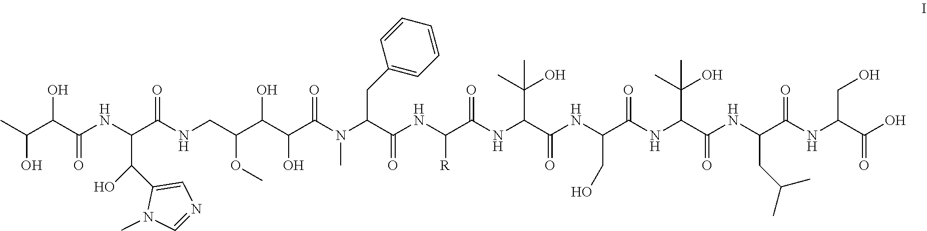

in which the group R is hydrogen or hydroxy, or a pharmaceutically acceptable salt of any of them.

Another subject of the present invention is a compound of the formula Ia,

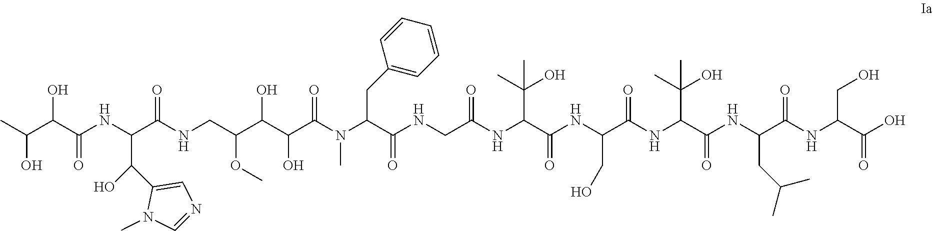 [SMILES: COC(CNC(=O)C(NC(=O)C(O)C(C)O)C(O)c1cncn1C)C(O)C(O)C(=O)N(C)C(Cc1ccccc1)C(=O)NCC(=O)NC(C(=O)NC(CO)C(=O)NC(C(=O)NC(CC(C)C)C(=O)NC(CO)C(=O)O)C(C)(C)O)C(C)(C)O]

i.e. a compound of the formula I in which the group R is hydrogen (H), or a pharmaceutically acceptable salt of any of them.

Another subject of the present invention is a compound of the formula Ib,

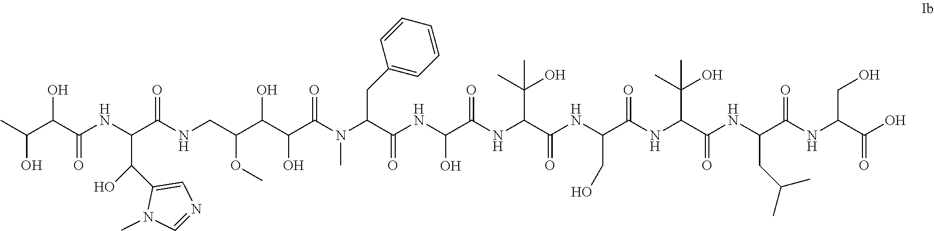 [SMILES: COC(CNC(=O)C(NC(=O)C(O)C(C)O)C(O)c1cncn1C)C(O)C(O)C(=O)N(C)C(Cc1ccccc1)C(=O)NC(O)C(=O)NC(C(=O)NC(CO)C(=O)NC(C(=O)NC(CC(C)C)C(=O)NC(CO)C(=O)O)C(C)(C)O)C(C)(C)O]

i.e. a compound of the formula I in which the group R is hydroxy (HO), or a pharmaceutically acceptable salt of any of them.

Another subject of the present invention is a compound of the formula Ic,

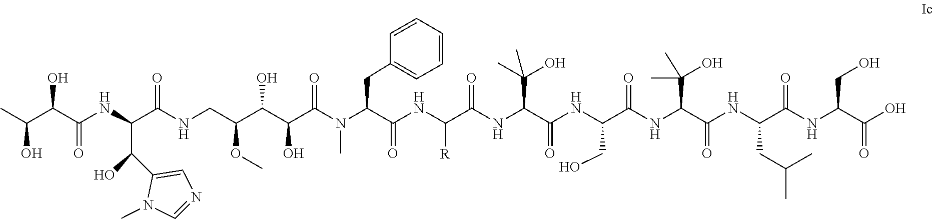

in which the group R is hydrogen or hydroxy, or a pharmaceutically acceptable salt of any of them.

Another subject of the present invention is a compound of the formula Id,

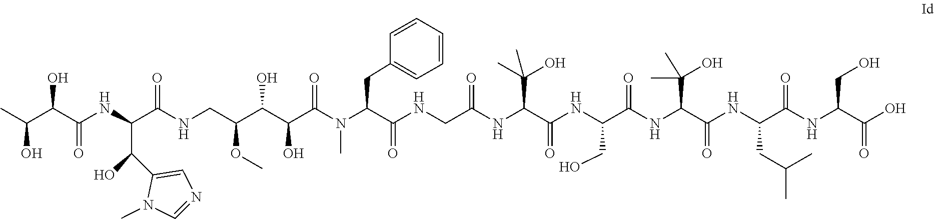 [SMILES: CO[C@@H](CNC(=O)[C@H](NC(=O)[C@H](O)[C@H](C)O)[C@H](O)c1cncn1C)[C@H](O)[C@H](O)C(=O)N(C)[C@@H](Cc1ccccc1)C(=O)NCC(=O)N[C@H](C(=O)N[C@@H](CO)C(=O)N[C@H](C(=O)N[C@@H](CC(C)C)C(=O)N[C@@H](CO)C(=O)O)C(C)(C)O)C(C)(C)O]

i.e. a compound of the formula Ic in which the group R is hydrogen, or a pharmaceutically acceptable salt of any of them.

Another subject of the present invention is a compound of the formula Ie,

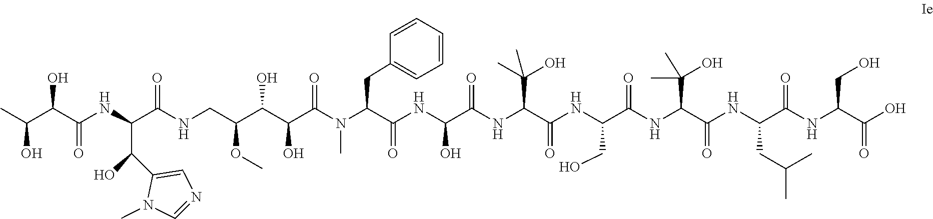 [SMILES: CO[C@@H](CNC(=O)[C@H](NC(=O)[C@H](O)[C@H](C)O)[C@H](O)c1cncn1C)[C@H](O)[C@H](O)C(=O)N(C)[C@@H](Cc1ccccc1)C(=O)N[C@H](O)C(=O)N[C@H](C(=O)N[C@@H](CO)C(=O)N[C@H](C(=O)N[C@@H](CC(C)C)C(=O)N[C@@H](CO)C(=O)O)C(C)(C)O)C(C)(C)O]

i.e. a compound of the formula Ic in which the group R is hydroxy, or a pharmaceutically acceptable salt of any of them.

Any reference to formula I is to be understood as including a reference to formulae Ia, Ib, Ic, Id and Ie, unless stated otherwise.

Pharmaceutically acceptable salts of the compounds of formula I do form part of the invention.

A further subject of the present invention is a process for the production of a compound of the formula I in which the group R is hydrogen or hydroxy, or a pharmaceutically acceptable salt of any of them,

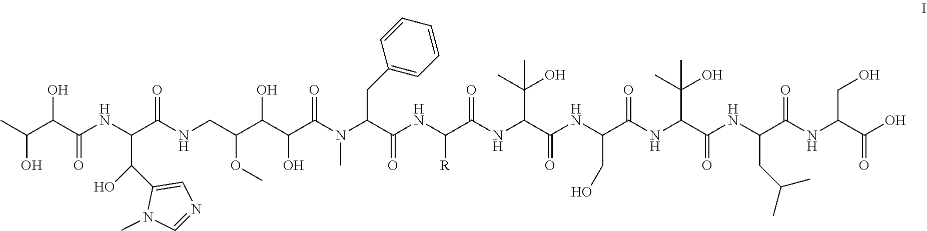

Another subject of the present invention is a process for the preparation of a compound of the formula I or a pharmaceutically acceptable salt thereof, wherein R is hydrogen or hydroxy, which comprises:
1) fermenting the microorganism strain ST201330 (DSM 24989) or one of its variants and/or mutants under suitable conditions in a culture medium,
2) isolating a compound of the formula I from the culture medium, and
3) optionally converting it into a pharmaceutically acceptable salt.

Another subject of the present invention is the microorganism strain ST201330 (DSM24989), described further below.

The processes according to the invention can be performed by conducting routine operations, for example in the fermentation step or in the isolation step, which are known to a person skilled in the art, and by using standard equipment. They can be performed on a laboratory scale, for example on a milliliter to liter scale, and on an industrial scale, for example on a cubic meter scale.

The culture medium, in which the fermentation step is performed, is an aqueous nutrient solution or a solid medium, for example a gel or agar, which contains at least one carbon source and nitrogen source, and generally further components such as customary inorganic salts. Suitable carbon sources for the fermentation are, for example, assimilable carbohydrates and sugar alcohols, such as glucose, lactose, sucrose, D-mannitol and carbohydrate-containing natural products like, for example, malt extracts, yeast extracts or oat flakes. Suitable nitrogen sources and in part also carbon sources are, for example, ammonium salts, nitrates, amino acids, peptides, including synthetically or biosynthetically obtained peptides, and proteins and their degradation products like, for example, Probion F (cf. K. Gerth et al., Applied Microbiology and Biotechnology 1984, vol. 19, p. 23-28), casein, peptone or tryptone, meat extracts, yeast extracts, gluten, ground seeds, for example of corn, wheat, beans, soybeans or cotton plant, distillation residues from alcohol production, or meat meals. Inorganic salts are, for example, chlorides, carbonates, sulfates or phosphates of alkali metals or alkaline earth metals like sodium, potassium, magnesium and calcium, and salts or other compounds of metals like iron, zinc, cobalt and manganese, which may be regarded as trace elements. Other components, which may be added in the fermentation step, are buffer substances, acids and bases for maintaining a certain pH range. As further components to liquid culture mediums, polymeric resins or other solid adsorbents, which adsorb fermentation products and may shift equilibria and facilitate work-up and isolation, for example Amberlite XAD resins like XAD-16, and/or customary antifoaming agents may be added.

An example of a suitable culture medium for the production of compounds of the formula I by fermentation of the microorganism strain ST201330 (DSM 24989) is a medium which contains from 0.05 to 5%, for example about 0.5%, of gluten; from 0.005 to 5%, for example about 0.5%, of glycerol; from 0.01 to 1%, for example about 0.1%, of CaCl₂)×2H₂O; from 0.01 to 1%, for example about 0.2%, of MgSO₄×7H₂O; and from 0.00001 to 0.001%, for example about 0.00005%, of cyanocobalamin; and favorably contains from 1% to 5%, for example about 2%, of the adsorbent resin XAD-16; and has a pH of from 7.5 to 8.0, for example about 7.8 (MEDIUM 1). Another example of such a culture medium is a medium which contains from 0.005 to 5%, for example about 0.5%, of oat flakes; from 0.05 to 5%, for example about 0.5%, of gluten; from 0.005 to 5%, for example about 0.5%, of glycerol; from 0.005 to 5%, for example about 0.5%, of glucose; from 0.01 to 1%, for example about 0.1%, of CaCl₂)×2H₂O; from 0.01 to 1%, for example about 0.2%, of MgSO₄×7H₂O; and from 0.00001 to 0.001%, for example about 0.00005%, of cyanocobalamin; and favorably contains from 1% to 5%, for example about 2%, of the adsorbent resin XAD-16; and has a pH of from 7.5 to 8.0, for example about 7.8 (MEDIUM 2). Unless specified otherwise, percentages relating to culture mediums which are given herein, generally are percentages by weight, based on the weight of the entire culture medium.

The culturing of the microorganism strain ST201330 (DSM 24989) is carried out aerobically, for example in submerse form with shaking or stirring in shaker flasks or fermenters, or on a solid medium, optionally with the introduction of air or oxygen. It can generally be carried out at temperatures from 18 to 35° C., for example from 20 to 32° C., in particular from 27 to 30° C. The pH can generally be between 4 and 10, for example between 6.5 and 9, in particular between 7.5 and 8.0. The microorganism is in general cultured under these conditions over a period of 2 to 18 days, for example 3 to 9 days. Advantageously, culturing is carried out in a number of stages, i.e., first, one or more precultures are prepared, for example in a liquid nutrient medium, that are then inoculated into the actual production medium, the main culture, for example in a volume ratio of 1:10 to 1:100. The preculture is obtained, for example, by inoculating the strain in the form of vegetative cells or fruiting bodies into a nutrient solution and allowing it to grow for 2 to 13 days, for example 4 to 10 days. Vegetative cells and/or fruiting bodies can be obtained, for example, by allowing the strain ST201330 (DSM 24989) to grow for 3 to 15 days, for example 7 to 10 days, on a solid or liquid nutrient medium, for example yeast agar.

An isolate of the microorganism strain with the internal identification number ST201330 was deposited by Sanofi-Aventis Deutschland GmbH, Industriepark Höchst, 65926 Frankfurt/Main, Germany, on Jul. 4, 2011 with DSMZ-Deutsche Sammlung von Mikroorganismen und Zellkulturen GmbH, meanwhile named Leibniz-Institut DSMZ-Deutsche Sammlung von Mikroorganismen und Zellkulturen GmbH (Leibniz Institute DSMZ-German Collection of Microorganisms and Cell Cultures GmbH), Inhoffenstraße 7B, 38124 Braunschweig, Germany, under accession number DSM 24989 in accordance with the Budapest Treaty on the International Recognition of the Deposit of Microorganisms for the Purposes of Patent Procedure.

The vegetative cells of the strain ST201330 (DSM 24989) have a characteristic rod shape. On solid nutrient media, ST201330 (DSM 24989) forms brownish fruiting bodies, which contain round myxospores. The taxonomy of the strain ST201330 can therefore be described as myxobacterium Corallococcus coralloides.

Instead of the strain ST201330 (DSM 24989), it is also possible to employ its mutants and/or variants which synthesize one or more of the compounds according to the present invention.

A mutant is a microorganism in which one or more genes of the genome have been modified, wherein the gene or the genes which are responsible for the ability of the organism to produce the compounds according to the present invention remain functional and inheritable. Mutants of this type can be produced in a manner known per se by physical means, for example irradiation, such as with ultraviolet or X-ray beams, or by treatment with chemical mutagens such as, for example, ethyl methanesulfonate (EMS), 2-hydroxy-4-methoxybenzophenone (MOB) or N-methyl-N′-nitro-N-nitrosoguanidine (MNNG), for example (cf., for example, T. D. Brock et al., Biology of Microorganisms, Prentice-Hall, Inc., 4^{th} ed., 1984, p. 305-315).

A variant is a phenotype of the microorganism. Microorganisms have the ability to adapt to their environment and therefore show marked physiological flexibility. In the case of phenotypic adaptation, all cells of the microorganism are involved, wherein the nature of the modification is not genetically conditioned and is reversible under modified conditions (cf., for example, H. Stolp, Microbial ecology: organisms, habitats, activities, Cambridge University Press, 1988, p. 180).

Screening for mutants and/or variants of the microorganism, which synthesize one or more of the compounds according to the invention, can be carried out, for example, by fermentation, lyophilization of the fermentation medium and extraction of the lyophilizate with an organic solvent, or extraction of the product compounds from the culture filtrate by adsorption to solid phases, and analysis by means of high performance liquid chromatography (HPLC) or thin layer chromatography (TLC), or by testing of the biological activity. The described fermentation conditions can be used for ST201330 (DSM 24989) and for mutants and/or variants thereof.

The isolation of the compounds of the formula I from the culture medium, and their purification, can be carried out according to general methods known to a person skilled in the art, taking into consideration the chemical, physical and biological properties of the formed natural products and the used microorganism. For determining the concentration of the fermentation products in the culture medium, for example for deciding on the length of the fermentation step, and during the individual isolation and purification stages, for example for characterizing a series of fractions, HPLC can favorably be used as analytical method.

In the isolation step that may also be regarded as an isolation and purification step, the culture broth can be centrifuged and/or filtered off through a suction filter. If the fermentation product is present in the liquid phase, further work-up after such removal of the microorganism can be performed by concentrating the liquid phase, for example by lyophilization, and/or extraction with organic solvents and/or mixtures of organic solvents and water. If the fermentation is performed in the presence of an adsorbent resin to which the fermentation product is adsorbed, centrifugation or filtration yields a mixture of the resin and the microorganism. For further work-up, the mixture can be lyophilized and the fermentation product extracted from the lyophilizate with organic solvents or mixtures of organic solvents and water. As organic solvents for the extraction of the compounds of the invention, alkanols like methanol or 2-propanol, are suitable, and may be also used in mixture with water. The obtained organic solvent phase which contains the natural products according to the invention, can be concentrated, generally in vacuo, for example by lyophilization, and subjected to further work-up and purification.

For further purification of the isolated fermentation product and separation of contained compounds from one another, favorably chromatographic method on suitable materials, for example on molecular sieves, alumina, silica gel, reversed phase silica gel (RP), ion exchangers or adsorbent resins can be used. As eluents in such chromatographic methods, organic solvents, water, buffered, neutral, basic or acidic aqueous solutions, or mixtures of water or aqueous solutions and organic solvents can be used, wherein suitable organic solvents preferably are miscible with water and buffered aqueous solutions, and suitable buffered aqueous solutions miscible with organic solvents, and wherein the content of the components of the eluent in such mixtures can range from 0 to 100%, and favorably a gradient is employed. Examples of organic solvents, which can be used in the chromatographic purification and separation of compounds of the present invention, are alkanols like methanol or 2-propanol, and acetonitrile. Examples of buffered, neutral, basic or acidified aqueous solutions are, for example, phosphate buffers, citrate buffers, ammonium acetate buffer or ammonium formate buffer of the desired pH in a concentration of up to about 0.5 M, and aqueous solutions of formic acid, acetic acid, trifluoroacetic acid, ammonia, triethylamine or other suitable acids and bases known to the person skilled in the art, generally in a concentration of up to about 1%. A favorable buffered aqueous solution for the chromatographic purification and separation of compounds of the present invention is 0.1% ammonium formate solution.

A purification and separation of compounds according to the invention on the basis of their differing polarity can be carried out with the aid of reversed phase chromatography, for example on hydrophobic materials such as, for example, RP-8 or RP-18 phases, or on MCI adsorber resin (Mitsubishi Chemical Company) or Amberlite XAD (Tosohaas). The separation can also be carried out with the aid of normal phase chromatography, for example on silica gel, alumina and the like. Also gel chromatography can be carried out, for example on polyacrylamide gels or mixed polymer gels such as, for example, Biogel P-2 (Biorad) or Fractogel TSK HW 40 (Merck). Chromatography for the purification and separation of the compounds according to the invention can be performed on several solid phases of different types and in different sequences. From mixtures of stereoisomers of the compounds according to the invention, individual stereoisomers can be separated by means of chromatography on a chiral solid phase, for example.

The compounds according to the invention, in particular the compounds of the formula Id, Ie and their pharmaceutically acceptable salts, exhibit antibacterial activities, in particular against Gram-negative bacteria, for example strains of Escherichia Coli. The biological activity of the compounds according to the invention can be shown by means of the tests described herein and other routine tests known to a person skilled in the art, for example a test for the detection of the antimicrobial activity of rapidly growing, aerobic causative organisms, such as the broth dilution method (microdilution), according to a procedure of the Clinical and Laboratory Standards Institute CLSI (cf. CLSI document M7-A7; vol. 26, no. 2). As a measure of the antibacterial activity, inhibitory concentrations like IC₈₀ values or IC₅₀ values that specify the concentration of a test compound that is needed for inhibiting the growth of the test organism by 80% or 50% of the maximal inhibition, and MIC values that specify the lowest concentration of a test compound that is needed for inhibiting growth of the test organism, may be given.

The compounds according to the present invention, in particular the compounds of the formula Ic, Id and Ie, can further be used as tool and/or aid in laboratory experiments and for diagnostic purposes.

The following examples illustrate the present invention in more detail, without restricting it in any way.

Percentages relate to the weight. Mixing ratios in the case of liquids relate to the volume, unless specified otherwise.

### EXAMPLE 1. STORAGE OF ST201330 (DSM 24989)

An agar plate (1% fresh baker's yeast; 0.1% CaCl₂)×2H₂O; 0.477% HEPES (4-(2-hydroxyethyl)-1-piperazineethanesulfonic acid; 20 mM); 0.00005% cyanocobalamin; 1.8% agar; pH 7.2) was inoculated with the strain ST201330 (DSM 24989) and incubated for 7 to 10 days at 30° C. The cells of this surface culture were scraped from the agar surface using a sterile spatula, suspended in 1 ml of Casitone medium (1% Casitone; Difco); 0.15% MgSO₄×7H₂O; 25% glycerin; pH 7.0) in cryotubes and stored at a temperature of either −135° C. or −196° C.

### EXAMPLE 2. PREPARATION OF A PRECULTURE OF ST201330 (DSM 24989)

100 ml of nutrient solution (1% fresh baker's yeast; 0.1% CaCl₂×2H₂O; 0.477% HEPES (20 mM); 0.00005% cyanocobalamin; pH 7.2) in a sterile 300 ml Erlenmeyer flask were inoculated with the strain ST201330 (DSM 24989) and incubated for 7 days at 30° C. and 180 rpm on a rotary shaker. 10 ml (10%) of this preculture were subsequently used for the inoculation of 100 ml main culture.

### EXAMPLE 3. PREPARATION OF A LIQUID MAIN CULTURE OF ST201330 (DSM 24989) (IN MEDIUM 1, AS DESCRIBED ABOVE)

A sterile 300 ml Erlenmeyer flask containing 100 ml of MEDIUM 1 was inoculated with 10 ml (10%) of a preculture (Example 2), or a culture grown on a fresh agar plate (1% fresh baker's yeast; 1% CaCl₂)×2H₂O; 0.477% HEPES (20 mM); 0.00005% cyanocobalamin; 1.8% agar; pH 7.2), and incubated on a shaker at 180 rpm and 30° C. The maximum production of the compounds of the formula I was reached after 96 to 216 hours.

### EXAMPLE 4. PREPARATION OF A LIQUID MAIN CULTURE OF ST201330 (DSM 24989) (IN MEDIUM 2, AS DESCRIBED ABOVE)

A sterile 300 ml Erlenmeyer flask containing 100 ml of MEDIUM 2 was inoculated with 10 ml (10%) of a preculture (Example 2), or a culture grown on a fresh agar plate (1% fresh baker's yeast; 1% CaCl₂×2H₂O; 0.477% HEPES (20 mM); 0.00005% cyanocobalamin; 1.8% agar; pH 7.2), and incubated on a shaker at 180 rpm and 30° C. The maximum production of the compounds of the formula I was reached after 96 to 216 hours.

### EXAMPLE 5. PREPARATION OF COMPOUNDS OF THE FORMULA I BY FERMENTATION OF ST201330 (DSM 24989) IN A FERMENTER

A 10 l fermenter was operated under the following conditions:
Inoculum: 10%
Nutrient medium: 0.5% gluten; 0.5% glycerol; 0.5% oat flakes; 0.5 glucose; 0.1% CaCl₂×2H₂O; 0.2% MgSO₄×7H₂O; 0.00005% cyanocobalamin; 2% of adsorber resin XAD-16; pH 7.8
Incubation temperature: 30° C.
Stirrer speed: 180 rpm
Aeration: 3 l/min
pH regulation: none, before sterilization pH 7.8±0.3 by means of KOH
pO₂ regulation: none
Antifoam additive: 0.05% Desmophen (Bayer)
Run time: 144 h

### EXAMPLE 6. ISOLATION OF THE COMPOUNDS OF THE FORMULA I FROM FLASK CULTURES OF ST201330 (DSM 24989)

After completion of the fermentation of the microorganism strain ST201330 (DSM 24989), the culture broth from Example 3 (45 l of combined culture broth from a series of fermentations) was separated by centrifugation. The biomass containing the adsorber resin XAD-16 was lyophilized and subsequently extracted with methanol/water (1:1; 3 times 4 l) and methanol/water (3:1; 2 times 4 l). The extracts were filtered and subsequently applied to a column which was filled with about 5.8 l of CHP-20P material (MCI® Gel, 75-150 μm, Mitsubishi Chemical Corporation). Elution was carried out using a 2-propanol gradient of from 5% to 40% over 60 min. The column flow (250 ml/min) was collected in 1 l fractions. Fractions 9 to 11 were combined, the solvent was removed on an evaporator and the fraction pool was subsequently lyophilized to give a residue of about 15 g.

### EXAMPLE 7. PRE-SEPARATION OF THE COMPOUNDS OF THE FORMULAE IA AND IB BY RP-18 CHROMATOGRAPHY

The residue of the pooled fractions 9 to 11 from Example 6 was dissolved in about 300 ml of methanol and applied in portions of about 20 ml (containing about 1 g each) to a Phenomenex Luna® 10 μm C18 (2) column (dimensions: 250 mm×50 mm) and eluted over a period of 40 min using a gradient of from 25% to 50% methanol in water containing 0.1% formic acid). The flow rate was 140 ml/min, the fraction size 140 ml. Fractions 21 to 24 of the individual runs were combined to pools 1 to 3 according to their purity which was determined by HPLC-UV-MS analysis, and subsequently worked up further.

### EXAMPLE 8. FINAL PURIFICATION OF THE COMPOUND OF THE FORMULA IA

Pool 1 and pool 3 from Example 7 were first evaporated and freeze-dried (yield from pool 1 about 690 mg, from pool 3 about 440 mg), then dissolved in 30 ml of methanol/water (1:1) and again purified by means of HPLC on a Phenomenex PhenylHexyl C18 10 μm column (dimensions: 100 mm×50 mm). Elution was carried out over a period of 40 min using a gradient of from 25% to 60% methanol in water (with addition of 50 g ammonium acetate per liter; pH 6.8 not adjusted). The column flow (140 ml/min) was collected in 190 ml fractions by time (80 sec). Fractions 22 to 24 from the separation of pool 1 and fractions 18 to 21 from the separation of pool 3 contained the compound of the formula Ia and afforded after lyophilization 230 mg of compound of the formula Ia.

### EXAMPLE 9. CHARACTERIZATION OF THE COMPOUND OF THE FORMULA ID (FORMULA GIVEN ABOVE)

White powder from acetonitrile/water after lyophilization
UV spectroscopy: end absorption
ESI(+) mass spectroscopy: MW=1183.5628
Empirical formula: C₅₁H₈₁N₁₁O₂₁

NMR data (chemical shifts δ in ppm) of the compound of the formula Id are given in Table 1. 1D-¹H spectra and 2D spectra were obtained on a Bruker AVANCE 700 MHz instrument operating at 700.2 MHz (¹H) and 176.1 MHz (¹³C), respectively. 1D-¹³C spectra were obtained on a Bruker AVANCE 500 MHz instrument operating at 500.3 MHz (¹H) and 125.8 MHz (¹³C), respectively. The concentration was c=15 mg/ml in d₆-DMSO. 3 μl of trifluoroacetic acid (TFA) were added to a sample of 9 mg dissolved in 600 μl d₆-DMSO. The temperature was 300 K (i.e. 26.85° C.).

TABLE 1

| Structural unit (cf. formula Ia) | Position | δ (¹H) | δ (¹³C) |
|---|---|---|---|
| 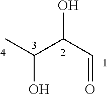 | 2 3 4 1 | 3.58 3.58 0.90 — | 75.12 67.50 19.38 172.06 |
| 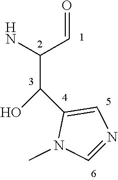 | NH 2 3 4 5 N—CH₃ 6 1 | 7.63 4.83 4.88 — 7.42 3.93 8.95 — | — 54.47 64.90 133.35 118.55 34.24 136.13 169.09 |
| 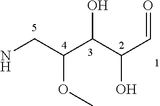 | NH 2 3 4 O—CH₃ 5 1 | 8.32 4.32 3.79 3.43 3.26 3.44/3.22 — | — 67.67 72.20 79.90 57.15 38.86 173.62 |
| 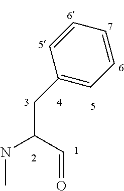 | N—CH₃ 2 3 4 5/5′ 6/6′ 7 1 | 2.92 5.28 3.34/2.88 — 7.24 7.24 7.17 — | 31.41 58.10 33.18 138.20 128.78 128.19 126.17 169.74 |
| 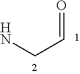 | NH 2 1 | 7.79 3.86/3.73 — | — 42.11 168.55 |
| 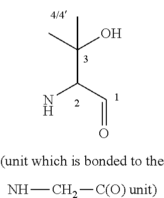 | NH 2 3 4/4′ 4/4′ 1 | 7.92 4.47 — 1.11 1.10 — | — 59.60 71.68 27.81 24.82 169.86 |
| 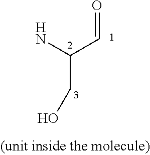 | NH 2 3 1 | 8.15 4.37 3.67/3.59 — | — 55.34 61.52 169.98 |
| 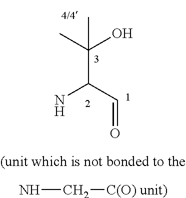 | NH 2 3 4/4′ 4/4′ 1 | 7.85 4.36 — 1.12 1.08 — | — 59.95 71.51 27.50 25.35 169.67 |
| 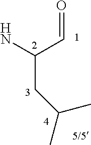 | NH 2 3 4 5/5′ 5/5′ 1 | 8.04 4.38 1.52/1.46 1.63 0.87 0.83 — | — 50.88 40.46 24.06 23.17 21.28 172.08 |
| 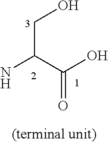 | NH 2 3 1 | 8.10 4.22 3.67/3.62 — | — 54.85 61.18 171.66 |

### EXAMPLE 10. FINAL PURIFICATION OF THE COMPOUND OF THE FORMULA IE

The initial pre-purification step and purification step as outlined in examples 7 and 8 are also suitable for the purification of the compound of the formula Ib. After evaporating and freeze-drying a pool containing both the compound of the formula Ia and the compound of the formula Ib from a chromatography which was carried out as described in example 7 (yield 30 mg), the resulting residue was dissolved in 3 ml of water and subsequently purified by means of HPLC on a PerfectSil 120 C8 10 μm column (dimensions: 250 mm×30 mm; injection in portions of 2 mg in 200 μl). For elution, a step-gradient was used. Within 1 min the flow of the initial eluent A (A=water/acetonitrile (98:2), +0.05% TFA) was increased from 5 to 30 ml/min. Then the eluent was adjusted to 70% eluent A and 30% eluent B (B=water/acetonitrile (50:50), +0.05% TFA), and these conditions were kept for 13 min. Afterwards the eluent was changed to 60% eluent A and 40% eluent B within 0.01 min and this ratio was kept for 9 min. Finally the eluent was adjusted within 3 min to 100% eluent B and these conditions kept for another 7 min. The column flow was collected based on UV absorption peaks (detection wavelength 205 nm). All fractions containing the compound of formula Ia were combined and freeze-dried, and the same was done with the fractions containing the compound of formula Ib. Evaporation and freeze-drying yielded 7.1 mg of the compound of the formula Ia and 5.9 mg of the compound of the formula Ib.

### EXAMPLE 11. CHARACTERIZATION OF THE COMPOUND OF THE FORMULA IE (FORMULA GIVEN ABOVE)

White powder from acetonitrile/water after lyophilization
UV spectroscopy: end absorption
ESI(+) mass spectroscopy: MW=1200.27
Empirical formula: C₅₁H₈₁N₁₁O₂₂

NMR data (chemical shifts δ in ppm) of the compound of the formula Ie are given in Table 2. 1D-¹H spectra and 2D spectra were obtained on a Bruker AVANCE 700 MHz instrument operating at 700.2 MHz (¹H) and 176.1 MHz (¹³C), respectively. 1D-¹³C spectra were obtained on a Bruker AVANCE 500 MHz instrument operating at 500.3 MHz (¹H) and 125.8 MHz (¹³C), respectively. The concentration was c=8.3 mg/ml in d₆-DMSO. 2 μl of TFA were added to a sample of 5 mg dissolved in 600 μl d₆-DMSO. The temperature was 300 K (i.e. 26.85° C.).

TABLE 2

| Structural unit (cf. formula Ib) | Position | δ (¹H) | δ (¹³C) |
|---|---|---|---|
| 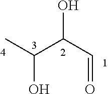 | 2 3 4 1 | 3.58 3.58 0.90 — | 75.12 67.52 19.40 172.14 |
| 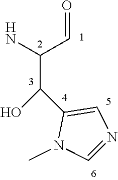 | NH 2 3 4 5 N—CH₃ 6 1 | 7.65 4.85 4.88 — 7.42 3.93 8.93 — | — 54.45 64.89 133.33 118.58 34.25 136.14 169.03 |
| 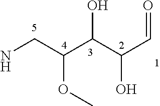 | NH 2 3 4 O—CH₃ 5 1 | 8.22 4.29 3.79 3.40 3.25 3.39/3.27 — | — 67.65 71.93 79.85 57.05 38.80 173.64 |
| 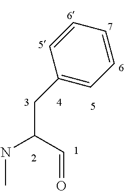 | N—CH₃ 2 3 4 5/5′ 6/6′ 7 1 | 2.94 5.44 3.33/2.88 — 7.25 7.25 7.17 — | 30.70 57.17 33.31 137.97 128.79 128.19 126.23 169.83 |
| 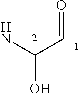 | NH 2 1 | 8.25 5.47 — | — 71.71 168.96 |
| 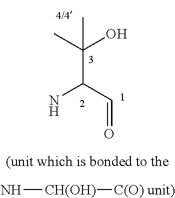 | NH 2 3 4/4′ 4/4′ 1 | 7.77 4.42 — 1.14 1.10 — | — 59.39 71.65 27.53 25.12 169.45 |
| 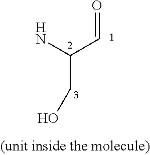 | NH 2 3 1 | 8.22 4.39 3.65/3.59 — | — 55.30 61.50 169.89 |
| 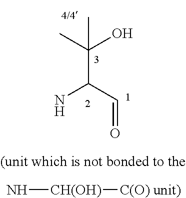 | NH 2 3 4/4′ 4/4′ 1 | 7.85 4.34 — 1.12 1.08 — | — 59.96 71.49 27.51 25.40 169.67 |
| 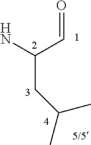 | NH 2 3 4 5/5′ 5/5′ 1 | 8.02 4.37 1.51/1.46 1.63 0.87 0.83 — | — 50.89 40.48 24.07 23.20 21.30 172.14 |
| 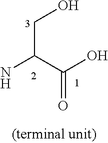 | NH 2 3 1 | 8.09 4.22 3.67/3.62 — | — 54.86 61.18 171.69 |

### EXAMPLE 12. DETERMINATION OF THE ANTIBACTERIAL ACTIVITY AGAINST ESCHERICHIA COLI: INHIBITORY ACTIVITY (1080)

### 12.1 Material and Methods

A stock solution of 1000 μg/ml of the test compound was prepared in methanol. The test strain (Escherichia coli ATCC 35218) was stored at −80° C. The inoculum was prepared from a fresh liquid preculture. The preculture was prepared from a bead of the material stored at −80° C. and 30 ml of nutrient medium (Mueller Hinton broth, Difco) and incubated at 37° C. and 180 rpm for 18 hours. The inoculum was adjusted to an optical density (OD) of 0.2 at a wavelength of 600 nm. For this, the inoculum was adjusted to a value of 10⁸ CFU/ml (CFU: colony forming units). After the adjustment of the inoculum, the suspension was diluted with nutrient solution (Mueller Hinton broth, Difco) in a ratio of 1:10000. The microtiter plate was inoculated within 15 min of preparation of the inoculum. The exact colony count was determined by means of surface culture. Using the stock solution of the test compound and the nutrient medium (Mueller Hinton broth, Difco), a dilution series was prepared beforehand on the microtiter plate. The test concentration of the test compound in the assays carried out was 64 to 0.125 μg/ml. The test compound was present in a volume of 20 μl and was treated with 20 μl of inoculum such that a total test volume of 40 μl was obtained. The inoculated microtiter plates were subsequently sealed with a lid and incubated at 37° C. in 5% CO₂ and at 95% atmospheric humidity for 20 hours. For each test, a control free from test compound, a sterile control and, as a reference substance, ciprofloxacin were co-tested on a 384 well microtiter plate. The microtiter plates were read with the aid of a photometer at a wavelength of 590 nm by measurement of the absorption. IC₈₀ values were subsequently calculated from the values of the dilution series according to a standard process as the concentration of the test compound which is necessary in order to inhibit the growth of the test organism Escherichia coli by 80%.

### 12.2 Results

The compound of the formula Ia had IC₈₀ values from 0.32 to 0.48 μg/ml, and the compound of the formula Ib had an IC₈₀ value of 0.71 μg/ml, for inhibition of Escherichia coli ATCC 35218.

### EXAMPLE 13. DETERMINATION OF THE ANTIBACTERIAL ACTIVITY AGAINST ESCHERICHIA COLI: MINIMAL INHIBITORY ACTIVITY (MIC)

### 13.1 Material and Methods

The present in vitro test identifies molecules having antimicrobial activity on the microorganisms by quantification of the culture optical density. Optical density compared to a vial with the same medium non inoculated by bacteria is an indicator of the bacterial growth.

Minimal inhibitory concentrations were evaluated following the CLSI and EUCAST guidelines. Bacterial inoculum were prepared by overnight culture in cation-adjusted Mueller-Hinton II (MHII) medium from colonies isolated from colonies on agar plates, then diluted to evaluate the colony forming units (cfu) using the McFarland turbidity standard. A 96 well plate was inoculated with 100 μl of 5.105 cfu/ml in MHII, and test compounds prepared by serial dilution at 100× concentration in DMSO. After a 20-22 h incubation, optical density was evaluated on a microplate reader and the MIC endpoint was determined as the lowest concentration of antibiotic at which there is no visible growth in duplicates, meaning that optical density differs no more from negative controls than 10% of the difference in optical density between negative controls (medium, no bacteria) and positive control (bacteria in medium, no antibiotics).

### 13.2 Results

The experiments performed demonstrate that the compounds according to the present invention have activity on inhibiting the growth of E. coli strain ATCC35218. The MIC values are typically between 1 and 8 μg/ml.

| Compound of | | Absorbance |
|---|---|---|
| formula | Number of experiments | MIC μg/mL |
| Id | 10 | 1-4 |
| Ie | 2 | 8 |

1-8. (canceled)

9. A compound of the formula I, 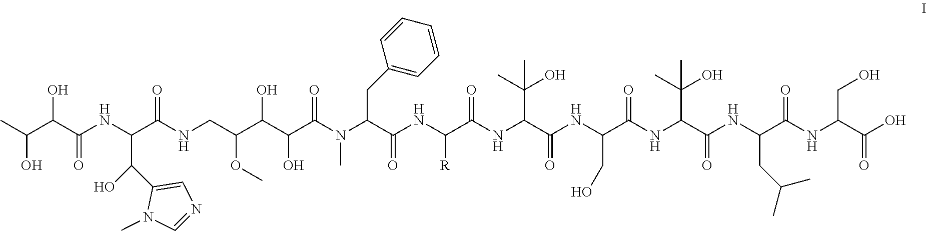
in which the group R is hydrogen or hydroxy,
or a pharmaceutically acceptable salt thereof.

10. A compound of the formula I according to claim 9 which is the compound of the formula Ia, 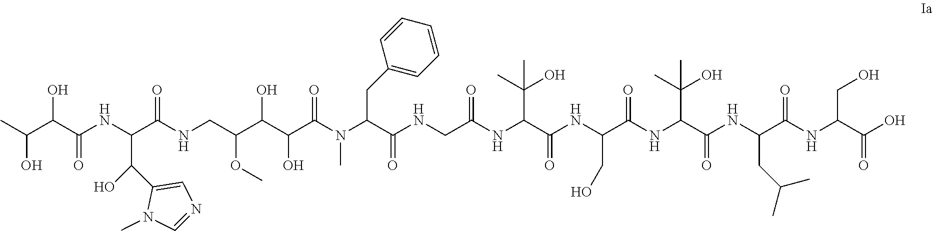 [SMILES: COC(CNC(=O)C(NC(=O)C(O)C(C)O)C(O)c1cncn1C)C(O)C(O)C(=O)N(C)C(Cc1ccccc1)C(=O)NCC(=O)NC(C(=O)NC(CO)C(=O)NC(C(=O)NC(CC(C)C)C(=O)NC(CO)C(=O)O)C(C)(C)O)C(C)(C)O]
or a pharmaceutically acceptable salt thereof.

11. A compound of the formula I according to claim 9 which is the compound of the formula Ib, 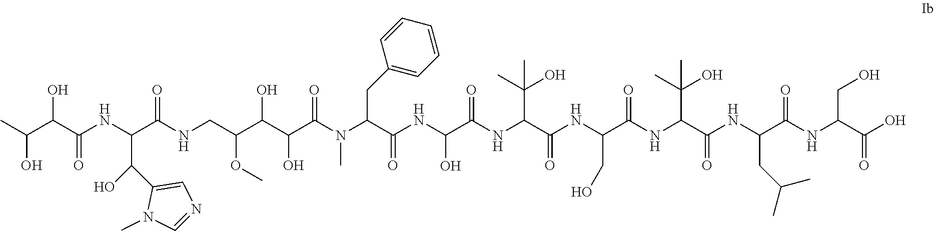 [SMILES: COC(CNC(=O)C(NC(=O)C(O)C(C)O)C(O)c1cncn1C)C(O)C(O)C(=O)N(C)C(Cc1ccccc1)C(=O)NC(O)C(=O)NC(C(=O)NC(CO)C(=O)NC(C(=O)NC(CC(C)C)C(=O)NC(CO)C(=O)O)C(C)(C)O)C(C)(C)O]
or a pharmaceutically acceptable salt thereof.

12. A compound of the formula I according to claim 9 which is the compound of the formula Ic, 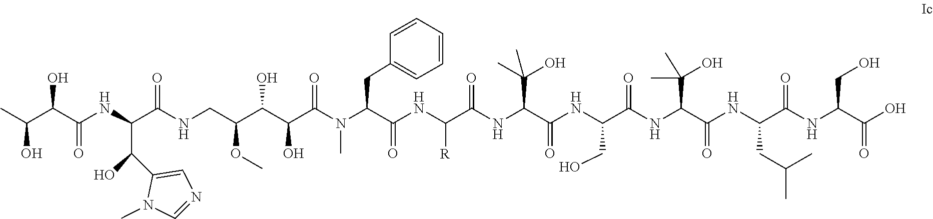
in which the group R is hydrogen or hydroxy,
or a pharmaceutically acceptable salt thereof.

13. A compound of the formula I according to claim 9 which is the compound of the formula Id, 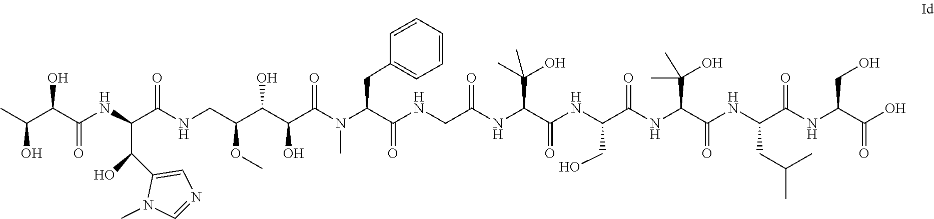 [SMILES: CO[C@@H](CNC(=O)[C@H](NC(=O)[C@H](O)[C@H](C)O)[C@H](O)c1cncn1C)[C@H](O)[C@H](O)C(=O)N(C)[C@@H](Cc1ccccc1)C(=O)NCC(=O)N[C@H](C(=O)N[C@@H](CO)C(=O)N[C@H](C(=O)N[C@@H](CC(C)C)C(=O)N[C@@H](CO)C(=O)O)C(C)(C)O)C(C)(C)O]
or a pharmaceutically acceptable salt thereof.

14. A compound of the formula I according to claim 9 which is the compound of the formula Ie, 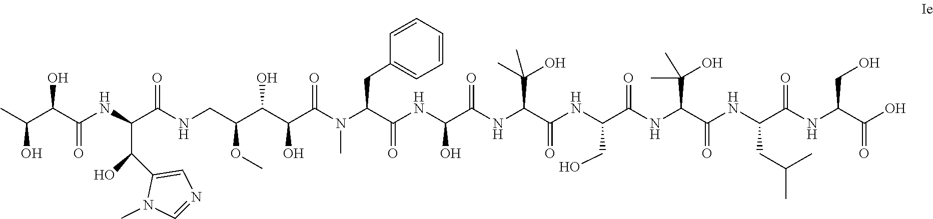 [SMILES: CO[C@@H](CNC(=O)[C@H](NC(=O)[C@H](O)[C@H](C)O)[C@H](O)c1cncn1C)[C@H](O)[C@H](O)C(=O)N(C)[C@@H](Cc1ccccc1)C(=O)N[C@H](O)C(=O)N[C@H](C(=O)N[C@@H](CO)C(=O)N[C@H](C(=O)N[C@@H](CC(C)C)C(=O)N[C@@H](CO)C(=O)O)C(C)(C)O)C(C)(C)O]
or a pharmaceutically acceptable salt thereof.

15. A process for the production of a compound of the formula I according to claim 9 or a pharmaceutically acceptable salt thereof, 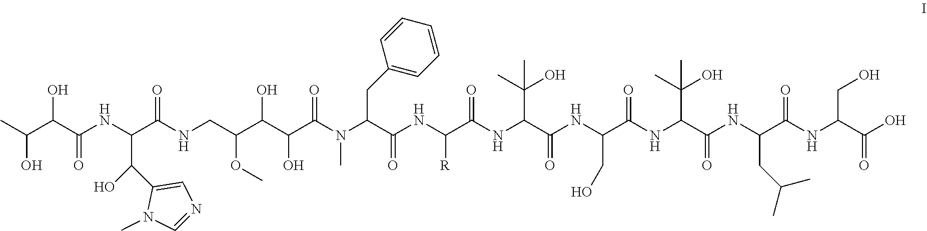
wherein the group R is hydrogen or hydroxy, which comprises
1) fermenting the microorganism strain ST201330 (DSM 24989) or one of its variants and/or mutants under suitable conditions in a culture medium, and
2) isolating a compound of the formula I from the culture medium.

16. A process for the production of a compound of the formula I according to claim 15, which further comprises 3) converting the compound of formula I into a pharmaceutically acceptable salt.

17. The microorganism strain ST201330 (DSM 24989).
